(19) United States
(12) Patent Application Publication
(10) Pub. No.: US 2004/0233971 A1
(43) Pub. Date: Nov. 25, 2004

(54) Temperature recording system

(76) Inventors: Roger W. Meads, Hortonville, WI (US); Scott Graunke, Colgate, WI (US)

Correspondence Address: J. Mitchell Jones, MEDLEN & CARROLL, LLP, 101 Howard Street, Suite 350, San Francisco, CA, 94105, US

(21) Appl. No.: 10/786,180

(22) Filed: Feb. 25, 2004

Related U.S. Application Data
(60) Provisional application No. 60/450,494, filed on Feb. 27, 2003.

(51) Int. Cl. G01K 1/00

(52) U.S. Cl. 374/208

(57) ABSTRACT

The present invention relates generally to temperature recording systems used in the monitoring of a large group of animals. In particular, the present invention relates to the general maintenance of a herd of dairy cows. The present invention eliminates many of the problems associated with the maintenance of dairy cows by providing a more efficient method of monitoring a dairy cow's body core temperature and by providing a more efficient way of locating a particular dairy cow.

### FIELD OF THE INVENTION

The present invention relates generally to temperature recording systems for monitoring the health status of a large group of animals. In particular, the present invention provides systems for monitoring the health, estrus status, and examination schedule for a herd of dairy cows. In preferred embodiments, the present invention utilizes RFID technology.

### BACKGROUND OF THE INVENTION

Dairy cow farming represents a major economic industry throughout the world. Most dairy farms operate with well over 100 dairy cows, and the daily maintenance required for each cow is significant. Two aspects of required daily maintenance for dairy cow farming is the monitoring of each cows temperature, and the ability to locate a desired cow within the herd.

Accurate and timely detection of a dairy cow's temperature is of major economic and health significance. For example, temperature monitoring permits the detection of a cow's estrus cycle. Early detection of an inflammation of the mammary gland which may lead to mastitis is possible with accurate temperature monitoring. Treatment of mastitis is suggested to cost $300 per incident, and as such, early detection prior to the onset of this disease saves a dairy farmer significant amounts of money. Avoidance and/or early treatment of other diseases is possible with accurate temperature monitoring, including respiratory disease, infectious contagious intestinal disease, and other bacterial, viral, and fungal diseases. Additionally, temperature monitoring permits detection of heat stress and subsequent avoidance of lower milk production, and reduced expression of estrus and conception rates.

The ability to locate a specific cow within the entire herd has major economic and health significance. Proper administration of medicine, vaccines, hormones, vitamins, and Bovines Somatotrophic Homone (BST) requires a dairy farmer to identify a specific cow within the herd. In addition, the dairy farmer must be able to identify within the herd which cows are entering estrus, exiting estrus, or about to enter estrus. Identification of specific cows within a herd is also necessary for cows to be induced into estrus, cows that are to have health maintenance procedures (e.g., treatment and care of feet and hooves), cows that are to be body scored, cows to be artificially inseminated, and among other reasons, cows that are to receive physical examinations.

Currently, dairy farmers monitor the temperature of each cow through the taking of daily rectal temperatures. The temperature is recorded based upon the identity of each cow. As such, daily temperature monitoring also serves as an identification process. While such monitoring serves the purpose of identification and health screening, the process itself is both time consuming and non-profitable.

Recurrent rectal temperature monitoring is difficult because determining the identity of the cow is time consuming. Cows typically are identified through neck chains or ear tags. Locating the neck chain or ear tag of a particular cow is often difficult because working areas have poor lighting or are situated in small areas. As such, the length of time required to take and monitor the temperature increases, and profitability decreases.

Recurrent rectal temperature monitoring is difficult because dairy cows are rarely situated in one convenient location. Dairy cows are constantly co-mingling and in motion. The average cow spends about 70% of its time during a 24 hour period lying down in a free stall or stanchion. The remaining 30% of the time is spent being milked, standing in a group waiting to be milked, eating and drinking with their heads near the ground, walking to the milking parlor and back to the barn or free stall, or just socializing. As such, tracking down a certain cow for rectal temperature monitoring requires a high amount of time to actually locate the cow within the herd.

Recurrent rectal temperature monitoring is difficult because it may lead to a decreased daily through-put of the herd's productivity. The taking of rectal temperatures before or after each cow is lead through a milking parlor often times slows down and disrupts the milking procedures. As noted, the process is lengthened because identification of the cow is compromised due to poor lighting and small areas, and often times certain cows are missed, leading to further delays. Average cow through-put is very important in milking dairies. The 24 hour average output by a dairy farm is crucial, and disrupting this output by taking rectal temperatures is a significant economic risk.

What is needed is a method of monitoring the daily temperature of a cow which is quick, accurate, and does not require the dairy farmer to personally identify each and every cow. What is also needed is a method of locating a specific cow within a herd which is quick, accurate, and does not require the dairy farmer to personally locate such a cow within the herd.

### SUMMARY OF THE INVENTION

The present invention relates generally to temperature recording systems form monitoring the health status of a large group of animals. In particular, the present invention provides systems for monitoring the health, estrus status, and examination schedule for a herd of dairy cows. In preferred embodiments, the present invention utilizes RFID technology.

Accordingly, in some embodiments, the present invention provides a system comprising an implantable temperature device, a signal receiver, a processor, a digital access device, and an animal identification device. In some embodiments, the system is used with animals. In preferred embodiments, the system is used with cows. In particularly preferred embodiments, the system is used to monitor the body core temperature of a cow.

In some embodiments, the implantable temperature device is implantable into an animal. In preferred embodiments, the implantable temperature device is implanted in the vulva of an animal. In some embodiments, the implantable temperature device contains a microchip. In preferred embodiments, the microchip contains a unique identification number.

In preferred embodiments, the system contains a signal receiver that is waterproof. In some embodiments, the signal receiver is positioned on a dairy farm. In preferred embodiments, the signal receiver is positioned in a milking parlor.

In preferred embodiments, the animal identification device is implanted on the exterior surface of an animal. In some embodiments, the animal identification device contains a power source. In preferred embodiments, the power source is manually changeable. In further embodiments, the animal identification device contains a signal device. In some embodiments, the signal device is an audible speaker. In preferred embodiments, the signal device is a visible light arrangement.

In some embodiments, the system contains a processor. In preferred embodiments, the processor runs algorithms. In further preferred embodiments, the algorithms integrate and process information received from the processor.

In preferred embodiments, the implantable temperature device, the signal receiver, and the processor transmit information through RFID technology. In further embodiments, the processor, the digital access device, and the animal identification device communicate with a wireless protocol. In particularly preferred embodiments, the wireless protocol is Bluetooth.

In other embodiments, the present invention provides a method of detecting estrus in an animal. In preferred embodiments the animal is a cow. In particular embodiments, the method provides a temperature recording system, a signal receiver, and a processor.

In preferred embodiments, the temperature recording system comprises at least one animal containing an implantable temperature device comprising a unique identification number. In other preferred embodiments, the animal also has an animal identification device, wherein the identification device comprises a signal device. In some preferred embodiments, the signal device comprises a light signal. In further preferred embodiments, the signal device illuminates in a distinctive manner. In even further preferred embodiments, the distinctive manner includes the illumination of a particular light bulb, or the flashing of a particular light bulb, or a combination of illumination and flashing of various colored light bulbs.

In some embodiments, the processor is a desktop computer. In preferred embodiments, the processor is located at a remote location, and the processor is accessed through a network. In preferred embodiments, the method further comprises the step of encoding the processor with a unique identification number for each animal. In other preferred embodiments, the method further comprises the step of encoding the processor with standardized animal temperature fluctuation data upon entry into estrus. In preferred embodiments, the method is used to detect the body core temperature of an animal with an implantable temperature sensor over an extended period of time. In some embodiments, the extended period of time is at least 1 hour and at most less than one year. In other preferred embodiments, fluctuations in the detected body core temperature are compared over an extended period of time. In further preferred embodiments, the method permits the identification of a particular animal entering estrus through transmittal of an estrus message from the processor to the animal identification device. In some embodiments, a digital access device facilitates transmittal of the estrus message. In preferred embodiments, the digital access device is a PDA. In some embodiments, the digital access device utilizes wireless technology. In preferred embodiments, the wireless technology is Bluetooth. In preferred embodiments, the estrus message illuminates the signal device in a distinctive manner. In further preferred embodiments, the distinctive manner includes the illumination of a particular light bulb, or the flashing of a particular light bulb, or a combination of illumination and flashing of various colored light bulbs.

In further embodiments, the method is used in developing a computer memory comprising a database. In even further embodiments, the method is used to develop a computer readable medium comprising a database.

In other embodiments, the present invention provides a method of maintaining an injection schedule for a herd of animals. In preferred embodiments the herd of animals is a herd of cows. In particular embodiments, the method provides a temperature recording system, a signal receiver, and a processor.

In preferred embodiments, the temperature recording system comprises at least one animal containing an implantable temperature device comprising a unique identification number. In other preferred embodiments, the animal also has an animal identification device, wherein the identification device comprises a signal device. In some preferred embodiments, the signal device comprises a light signal. In further preferred embodiments, the signal device illuminates in a distinctive manner. In even further preferred embodiments, the distinctive manner includes the illumination of a particular light bulb, or the flashing of a particular light bulb, or a combination of illumination and flashing of various colored light bulbs.

In some embodiments, the processor is a desktop computer. In preferred embodiments, the processor is located at a remote location, and the processor is accessed through a network. In preferred embodiments, the method further comprises the step of encoding the processor with a unique identification number for each animal. In other preferred embodiments, the method further comprises the step of encoding the processor with an injection schedule for each animal. In further preferred embodiments, the injection schedule includes, but is not limited to, hormonal injections, immunization injections, and BST injections.

In further preferred embodiments, the present invention provides methods of maintaining an injection schedule for a herd of animals comprising the steps of: a) providing a temperature recording system comprising: i) at least one animal containing an implantable temperature device comprising a unique identification number; said at least one animal having an animal identification device, wherein said identification device comprises an signal device, ii) a signal receiver, and iii) a processor, and b) identifying a particular animal scheduled for an injection event through transmission of an injection message from said processor to said animal identification device. In some embodiments, a digital access device facilitates transmittal of the injection message. In preferred embodiments, the digital access device is a PDA. In some embodiments, the digital access device utilizes wireless technology. In preferred embodiments, the wireless technology is Bluetooth. In preferred embodiments, the injection message illuminates the signal device in a distinctive manner. In further preferred embodiments, the distinctive manner includes the illumination of a particular light bulb, or the flashing of a particular light bulb, or a combination of illumination and flashing of various colored light bulbs.

In other embodiments, the present invention provides methods of maintaining a health check-up schedule herd of animals comprising a) providing a temperature recording system comprising: i) at least one animal containing an implantable temperature device comprising a unique identification number; said at least one animal having an animal identification device, wherein said identification device comprises an signal device, ii) a signal receiver, and iii)a processor, and b) identifying a particular animal scheduled for an injection event through transmission of a health check-up message from said processor to said animal identification device. In preferred embodiments the herd of animals is a herd of cows. In particular embodiments, the method provides a temperature recording system, a signal receiver, and a processor.

In preferred embodiments, the temperature recording system comprises at least one animal containing an implantable temperature device comprising a unique identification number. In other preferred embodiments, the animal also has an animal identification device, wherein the identification device comprises a signal device. In some preferred embodiments, the signal device comprises a light signal. In further preferred embodiments, the signal device illuminates in a distinctive manner. In even further preferred embodiments, the distinctive manner includes the illumination of a particular light bulb, or the flashing of a particular light bulb, or a combination of illumination and flashing of various colored light bulbs.

In some embodiments, the processor is a desktop computer. In preferred embodiments, the processor is located at a remote location, and the processor is accessed through a network. In preferred embodiments, the method further comprises the step of encoding the processor with a unique identification number for each animal. In other preferred embodiments, the method further comprises the step of encoding the processor with a health check-up schedule for each animal. In further preferred embodiments, the health check-up schedule includes, but is not limited to, veterinary examinations, feet trimming, mating, and body scoring.

In further preferred embodiments, the method permits the identification of a particular animal entering scheduled for an injection through transmittal of a health check-up message from the processor to the animal identification device. In some embodiments, a digital access device facilitates transmittal of the health check-up message. In preferred embodiments, the digital access device is a PDA. In some embodiments, the digital access device utilizes wireless technology. In preferred embodiments, the wireless technology is Bluetooth. In preferred embodiments, the health check-up message illuminates the signal device in a distinctive manner. In further preferred embodiments, the distinctive manner includes the illumination of a particular light bulb, or the flashing of a particular light bulb, or a combination of illumination and flashing of various colored light bulbs.

In other embodiments, the present invention provides methods of detecting the state of health in an animal comprising the steps of: a) providing a temperature recording system comprising: i) at least one animal containing an implantable temperature device comprising a unique identification number; said at least one animal having an animal identification device, wherein said identification device comprises an signal device, ii) a signal receiver, iii) a processor, and b) detecting body core temperature of said at least one animal with said implantable temperature device over an extended period of time; c) comparing said animal's body core temperature fluctuation over said extended period of time; and d) identifying a particular animal's state of health through transmittal of a state of health message from said processor to said animal identification device. In preferred embodiments the animal is a cow. In particular embodiments, the method provides a temperature recording system, a signal receiver, and a processor.

In preferred embodiments, the temperature recording system comprises at least one animal containing an implantable temperature device comprising a unique identification number. In other preferred embodiments, the animal also has an animal identification device, wherein the identification device comprises a signal device. In some preferred embodiments, the signal device comprises a light signal. In further preferred embodiments, the signal device illuminates in a distinctive manner. In even further preferred embodiments, the distinctive manner includes the illumination of a particular light bulb, or the flashing of a particular light bulb, or a combination of illumination and flashing of various colored light bulbs.

In some embodiments, the processor is a desktop computer. In preferred embodiments, the processor is located at a remote location, and the processor is accessed through a network. In preferred embodiments, the method further comprises the step of encoding the processor with a unique identification number for each animal. In other preferred embodiments, the method further comprises the step of encoding the processor with standardized animal temperature fluctuation data upon entry into infection. In some embodiments, the types of infection include, but are not limited to, general infection, respiratory disease, infectious contagious intestinal disease, viral infection, bacterial infection, and fungal infection.

In preferred embodiments, the method is used to detect the body core temperature of an animal with an implantable temperature sensor over an extended period of time. In some embodiments, the extended period of time is at least 1 hour and at most less than one year. In other preferred embodiments, fluctuations in the detected body core temperature are compared over an extended period of time. In further preferred embodiments, the method permits the identification of a particular animal entering a change of state of health through transmittal of a state of health message from the processor to the animal identification device. In some embodiments, a digital access device facilitates transmittal of the state of health message. In preferred embodiments, the digital access device is a PDA. In some embodiments, the digital access device utilizes wireless technology. In preferred embodiments, the wireless technology is Bluetooth. In preferred embodiments, the estrus message illuminates the signal device in a distinctive manner. In further preferred embodiments, the distinctive manner includes the illumination of a particular light bulb, or the flashing of a particular light bulb, or a combination of illumination and flashing of various colored light bulbs.

In further embodiments, the method is used in developing a computer memory comprising a database. In even further embodiments, the method is used to develop a computer readable medium comprising a database.

In other embodiments, the present invention provides a method of locating an animal within an animal herd. In preferred embodiments the herd of animals is a herd of cows. In particular embodiments, the method provides a temperature recording system, a signal receiver, and a processor.

In preferred embodiments, the temperature recording system comprises at least one animal containing an implantable temperature device comprising a unique identification number. In other preferred embodiments, the animal also has an animal identification device, wherein the identification device comprises a signal device. In some preferred embodiments, the signal device comprises a light signal. In further preferred embodiments, the signal device illuminates in a distinctive manner. In even further preferred embodiments, the distinctive manner includes the illumination of a particular light bulb, or the flashing of a particular light bulb, or a combination of illumination and flashing of various colored light bulbs.

In some embodiments, the processor is a desktop computer. In preferred embodiments, the processor is located at a remote location, and the processor is accessed through a network. In preferred embodiments, the method further comprises the step of encoding the processor with a unique identification number for each animal.

In further preferred embodiments, the method permits the identification of a particular animal through transmittal of a location message from the processor to the animal identification device. Accordingly, in some embodiments, the present invention provides methods of locating an animal within an animal herd comprising the steps of: a) providing a temperature recording system comprising: i) at least one animal containing an implantable temperature device comprising a unique identification number; said at least one animal having an animal identification device, wherein said identification device comprises an signal device, ii) a signal receiver, and iii) a processor, and b) identifying a particular animal through transmission of a location message from said processor to said animal identification device. In some embodiments, a digital access device facilitates transmittal of the location message. In preferred embodiments, the digital access device is a PDA. In some embodiments, the digital access device utilizes wireless technology. In preferred embodiments, the wireless technology is Bluetooth. In preferred embodiments, the location message illuminates the signal device in a distinctive manner. In further preferred embodiments, the distinctive manner includes the illumination of a particular light bulb, or the flashing of a particular light bulb, or a combination of illumination and flashing of various colored light bulbs.

### DESCRIPTION OF THE FIGURES

FIG. 1 depicts a schematic of the Temperature Recording System.

FIG. 2 depicts a schematic of RFID technology.

FIG. 3 depicts an animal identification device embodiment.

### DEFINITIONS

To facilitate the understanding of the invention, a number of terms are defined below.

As used herein, the term “implantable temperature device” generally refers to temperature sensing devices which operate with or without a separate power source. This term includes passive implantable temperature devices. Specific examples of passive implantable temperature devices include, but are not limited to, RFID reading devices, WFID reading devices, RFID transponders, and transponders.

As used herein, the term “signal receiver” refers to a devices capable of receiving information sent from an implantable temperature device. Examples include, but are not limited to, an RFID antenna and a standard radio frequency antenna.

As used herein, the term “processor” refers to a device capable of executing a computer algorithm and/or storing information to a computer memory.

As used herein, the term “algorithm” refers to computer programs. Specific examples include, but are not limited to, temperature computer programs, infection computer programs, and analytical computer programs.

As used herein, the term “cow” refers to any bovine species of any age (e.g., from birth to death) of any gender.

As used herein, the term “digital access device” refers to personal digital assistants. The digital access device may or may not utilize wireless technology such as Bluetooth or Wi-Fi.

As used herein, the term “animal identification device” refers generally to a collar or tag used in the wireless labeling of a subject. Specific examples include, but are not limited to, RFID tags, and RFID collars.

As used herein, the terms “RFID” or “RFID technology” refer generally to radio frequency identification devices.

As used herein, the term “core body temperature” refers to the internal body temperature of an animal.

As used herein, the term “power source” refers to the energy source used to power the transponder. A specific example includes, but is not limited to, batteries.

As used herein, the term “signal device” refers to a part of part of an animal identification device that displays or emits a signal. Specific examples include, but are not limited to, audio speakers emitting particular sounds, light devices emitting certain light displays, and text based messages.

As used herein, the term “temperature sensor” refers to a device capable of detecting the core body temperature of an animal. Specific examples include, but are not limited to, ISO thermometers, manual thermometers, and digital thermometers.

As used herein, the term “estrus” refers to the portion or phase of the sexual cycle of female animals characterized by a willingness to permit coitus.

### DETAILED DESCRIPTION

The present invention provides devices that embody aspects of a temperature recording system. The present invention also provides temperature monitoring systems and methods for using such temperature monitoring systems. The illustrated and preferred embodiments discuss these structures and techniques in the context of temperature recording devices used with cows. These structures, systems, and techniques are well suited for use with any type of animal within any type of setting.

The present invention relates to methods and compositions for the ascertaining and monitoring of subject temperatures across a group setting. In particular, the present invention utilizes RFID technology in the monitoring of dairy cow internal body core temperatures within a herd. In addition, the present invention contemplates methods of maintaining the health of a herd of dairy cows based upon such temperature monitoring. For convenience, the description of the invention is presented in the following sections: I) the temperature recording system; and II) uses of the temperature recording system.

I. Temperature Recording System

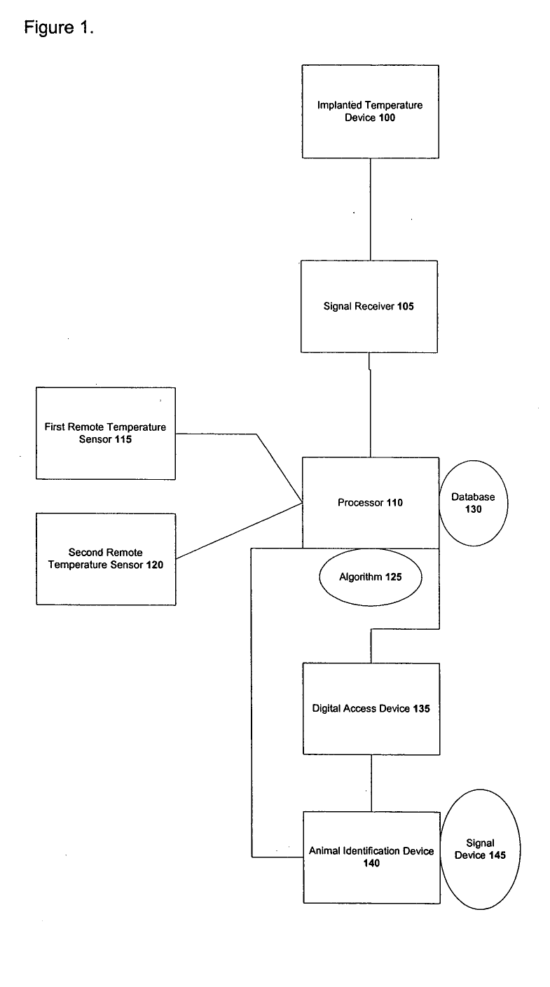

FIG. 1 presents a schematic depiction of an embodiment of temperature recording system. The core body temperature of an animal (e.g., livestock, human, cat, dog, goat, or any mammal) is measured with an implantable temperature device 100. In preferred embodiments, the core body temperature of an animal is measured with an implantable temperature device 100. In some preferred embodiments, the implantable temperature device 100 is implanted in female animals near the animal's vulva. In other preferred embodiments, the implantable temperature device 100 is implanted into the ear or eyelid of the animal. In preferred embodiments, the implantable temperature device 100 contains a unique identification number. In particularly preferred embodiments, the implantable temperature device 100 communicates core body temperature data and an identification number to a signal receiver 105 (e.g., an RFID antenna). In further preferred embodiments, the signal receiver 105 communicates this message to a processor 110.

In preferred embodiments, the processor 110 receives information from the signal receiver 105, and optionally, at least one remote temperature sensor (e.g., 115 and 120). The first remote temperature sensor 115 and the second remote temperature sensor 120 provide an ambient temperature reading, for example, from a barn or other animal loafing area.

In some embodiments, the processor 110 runs a temperature algorithm 125 stored on a computer readable medium (not shown). In such embodiments, the temperature algorithm 125 interprets the temperature readings provided by the implantable temperature device 100, the first remote temperature sensor 115, and the second remote temperature sensor 120. In further embodiments, the temperature algorithm 125 stores this information in a database 130. In preferred embodiments, the temperature algorithm 125 is programmed with certain temperature guidelines. For example, the temperature algorithm 125 is capable of interpreting the temperature fluctuations of a particular animal (e.g., dairy cow) over an extended period of time and determine if the animal is entering estrus.

In further preferred embodiments, the temperature algorithm 125 is also capable of assessing the presence of infection, heat stress, and other events. If the temperature algorithm 125 determines that a particular animal (e.g., dairy cow) is experiencing an event (e.g., estrus, infection, heat stress), the animal identification device 140 is alerted in a particular manner (e.g., dairy cow has infection; dairy cow is entering estrus). In further embodiments, the temperature algorithm 125 sends a message via the processor 110 to a digital access device (e.g., PDA, antenna) 135 which in turn relays the message to the animal identification device 140. In some embodiments, the processor 110 may send such a message directly to the animal identification device 140.

Each item discussed above in relation to FIG. 1 is more thoroughly described below as alternative and preferred embodiments.

A. Radio Frequency Identification (RFID)

In preferred embodiments, the present invention utilizes wireless communication technology (e.g., radio frequency identification devices). Electronic identification devices and systems have provided a good method for providing identification of animal. Typically, electronic identification systems utilize a passive electronic identification device that is induced to transmit its identification signal by an externally radiating source. These passive electronic identification devices may be a transponder carried with the individual animal on a collar as illustrated and described in Carroll U.S. Pat. No. 4,475,481, issued Oct. 9, 1984, entitled “Identification System” and in Kuzara U.S. Pat. No. 4,463,353, issued Jul. 31, 1984, entitled “Animal Feeding and Monitoring System”; in an ear tag such as those commercially available from Destron/Fearing, Inc., Allflex USA, Inc., and Avid Marketing, Inc.; in a transponder implanted in the animal as illustrated and described in Pollack U.S. Pat. No. 4,854,328, issued Aug. 8, 1989, entitled “Animal Monitoring Telltale and Information System” and in Hanton U.S. Pat. No. 4,262,632, issued Apr. 21, 1981, entitled “Electronic Livestock Identification System”; or in a bolus such as illustrated and described in U.S. Pat. No. 4,262,632, issued Apr. 21, 1981, entitled “Electronic livestock identification system” by John P. Hanton and Harley A. Leach.

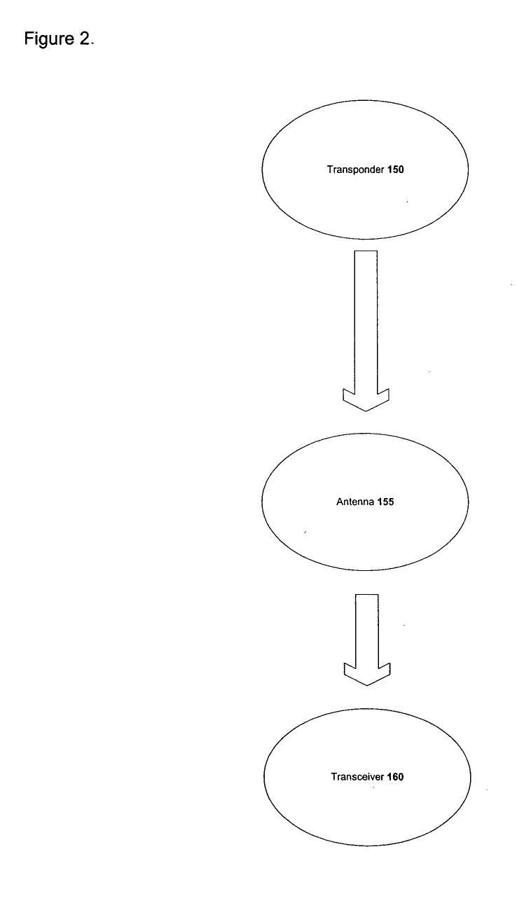

In some embodiments, the present invention utilizes radio frequency identification devices (RFID). FIG. 2 schematically illustrates the components of RFID systems which generally consist of a transponder 150, an antenna 155, and a transceiver 160. Briefly, RFID systems permit the wireless transfers of information between locations. For example, a transponder 150 is implanted into an animal (e.g., dairy cow). The transponder is able to detect information (e.g., identification number and temperature) within an animal and transmit this information to an antenna 155. Next, the antenna 155 relays this information to a transceiver 160. The transceiver 160 integrates this information, and processes the information. Transmission of information typically occurs in radio frequencies. The antenna 155 and transceiver can be a single unit (e.g., a signal receiver 105). The transceiver interfaces with the processor 110.

B. Implantable Temperature Device

In preferred embodiments, radio frequency identification transponders may be placed on an animal's ear or other portions of the animal's body. In further embodiments, transponders are generally passive devices that operate without a separate power source. When used to identify an animal, these devices contain a unique code for a particular animal.

When transponders are read using a radio frequency identification reader, they provide the unique code for that animal's identification. Several RFID readers are commercially available, typically from the transponder suppliers, including models from Destron/Fearing, Inc., Allflex USA, Inc., and Avid Marketing, Inc.

RFID readers that can distinguish multiple types of RFID transponders as illustrated and described in U.S. Pat. No. 5,235,326, issued Aug. 10, 1993, entitled “Multi-mode Identification System” to Michael L. Beigel, Nathaniel Polish, and Robert E. Malm. Another such reader is that illustrated and described in U.S. Pat. No. 5,952,935, issued Sep. 14, 1999, entitled “Programmable Channel Search Reader” to E. Zeke Mejia and Ian Griffiths.

In some embodiments, the present invention utilizes an implantable temperature device 100. Such an implantable temperature device 100 (referring to FIG. 1) is contemplated to meet industry standard RFID technology, and is a battery less design. In addition, the implantable temperature device is contemplated to operate only when within a specified antenna 155 range. The implantable temperature device 100 is contemplated to emit a very short radio signal (e.g., less than 100 m). In addition, in preferred embodiments, the implantable temperature device 100 contains a unique signature (e.g., 48 bit digital unique signature). For example, a number of suitable temperature sensing devices are known in the art, including, but not limited to the Cattle Temperature RFID bolus from Phase IV Engineering (Boulder, Colo.), the MAGIIX Bolus (Post Falls, Id.), and the BIOTHERM device from Digital Angel Corp (St. Paul, Minn.).

An animal's rectal, auricle, or vulval temperature reflects its internal core temperature. One function of the present invention is to ascertain animal's internal core body temperature. In some embodiments, an implantable temperature device 100 is configured to ascertain the core body temperature of an (e.g., dairy cow). In preferred embodiments of the present invention, the implantable temperature device 100 is implanted into an animal (e.g., goat). In preferred embodiments, the animal is a dairy cow. In preferred embodiments, implantation occurs within a surgical setting by a professional (e.g., medical doctor, veterinary medicine doctor). In other embodiments, implantation may occur with an implantation gun. The location of implantation is anywhere in an animal's body which will allow physical access from outside the animal's body, and is capable of ascertaining the animal's core body temperature. In preferred embodiments, an implanted temperature device 100 may remain in place beyond 4 years. In some embodiments, implantation is under the animal's lip of the ventral commissure of the vulva. In other embodiments, the implantable temperature device 100 is in the animal's rectum, eyelid, or ear. In further preferred embodiments, the implantable temperature device 100 is implanted in such a manner so as to facilitate easy retrieval.

In preferred embodiments, the present invention ascertains the body core temperature of an animal (e.g., dairy cow) with an implantable temperature device 100 configured with a temperature sensor. In preferred embodiments the implantable temperature device 100 is implanted into the vulva. In other preferred embodiments, the present invention utilizes the ingestible animal temperature sensor described in U.S. Pat. No. 6,371,927. The present invention is not limited by the use of any type of temperature sensor (e.g., manual electric thermometer, manual non-electric thermometer). In preferred embodiments, the temperature sensor meets ISO Standard temperature sensor qualifications for ascertaining an animal's (e.g., dairy cow) internal core body temperature. The present invention is not limited by where an implantable temperature device 100 configured with a temperature sensor is implanted within an animal.

In preferred embodiments, the implantable temperature device 100 equipped with a temperature sensor sends core body temperature and unique tag identification information to the signal receiver 105.

C. Signal Receiver

As discussed above, in preferred embodiments, the present invention implements wireless technology (e.g., RFID, WiFi) in the ascertaining and monitoring of an animal's internal core temperature. In further embodiments, the signal receiver 105 emits radio signals to activate the implantable temperature device 100, read data from the implantable temperature device 100, and relay data from the implantable temperature device 100 to the processor 110. In preferred embodiments, the present invention uses standard RFID signal receivers 105 (e.g., antennae). The present invention is not limited to a certain signal receiver 105 location. In some embodiments, a signal receiver 105 is located at the entrance to a barn. In preferred embodiments, a signal receiver 105 is located in the vicinity of a milking parlor. In further embodiments, the signal receiver 105 is waterproof, is configured to withstand erratic behavior from an animal (e.g., dairy cow), is small and unobtrusive, and is activated by a 110 electrical source.

In some signal receiver 105 embodiments, an antenna 155 emits radio waves in range of anywhere from 1 mm to more than 200 feet. In further embodiments, when an implantable temperature device 100 passes through a signal receiver's 105 electromagnetic zone, the implantable temperature device 100 detects a signal receiver 105 activation signal. The signal receiver 105 decodes the data encoded in the implantable temperature device's integrated circuit (e.g., microchip) and the data is passed to the processor 110. In preferred embodiments, the signal receiver 105 decodes temperature data from an implantable temperature device 100 within an animal and sends this information to a processor 110.

D. Processor

In preferred embodiments, a processor 110 (e.g., computer) receives information ascertained by the implantable temperature device 100, the first remote temperature sensor 115, and the second remote temperature sensor 120. The processor 110 interprets this information. The processor 110 also sends signals to the animal identification device 140 directly or via a digital access device 135. The present invention contemplates processors 110 consisting of various algorithms 125 (e.g., computer programs) and a large database 130.

In preferred embodiments, the processor 110 is located in a central location on a farm. In further embodiments, the processor 110 is located in a barn. In further embodiments, the processor 110 is networked with various algorithms 125 and the database 130. In further embodiments, the processor 110 interfaces with the signal receiver 105 with a physical connection (e.g., electrical wire). In other preferred embodiments, the processor 110 interfaces with the signal receiver 105 utilizing wireless technology (e.g., RFID, Bluetooth). In further embodiments, the processor 110 interfaces with the digital access device 135 with a physical connection (e.g., uploading of information onto a PDA). In other preferred embodiments, the processor 110 interfaces with the digital access device 135 with a wireless connection (e.g., Bluetooth). In other preferred embodiments, the processor is configured to run dairy management programs. In further preferred embodiments, the processor 110 is configured to run Dairy Comp 305.

In some embodiments, the present invention uses at least one remote temperature sensor (e.g., 115 and 120) to measure the ambient (e.g., environmental) temperature. In preferred embodiments, the present invention uses two remote temperature sensors. Typically, remote temperature sensors are positioned in a manner conducive for accurately measuring ambient temperature. In preferred embodiments, remote temperature sensors are located inside and outside of a frequent animal (e.g., dairy cow) location (e.g., inside and outside of barn; inside and outside of milking parlor). The present invention is not limited to any type of remote temperature sensor. In preferred embodiments, remote temperature sensors are ISO standard remote temperature sensors. The present invention contemplates remote temperature sensors capable of communicating temperature information with the processor 110 utilizing wireless technology (e.g., RFID).

The present invention is not limited to a particular type or kind of processor 110 (e.g., computer technology) in the archiving, processing, and interpretation of incoming animal data. In preferred embodiments, the processor 110 uses algorithms 125 to interpret temperature information. In some embodiments, temperature algorithms are used to detect dairy animal (e.g., cow) estrus, and to detect various infections or diseases (e.g., general infection, respiratory disease, infectious contagious intestinal disease, bacterial infection, fungal infection, viral infection, heat stress). In other preferred embodiments, the processor 110 uses algorithms 125 in the maintenance of a herd of dairy animals (e.g., dairy cows). In some embodiments, maintenance algorithms 125 are used to identify particular animals scheduled for maintenance (e.g., hoof trimming, veterinary checkup, physical examination, scheduled reproduction, hormone injections). The processor 110 stores information interpreted with various algorithms 125 in a database 130 contained on a computer readable medium (e.g., hard drive).

E. Digital Access Device

In preferred embodiments, a digital access device 135 is used as the interface between the processor 12 and the animal identification device 17. In further preferred embodiments, the digital access device 135 is a personal digital assistant (e.g., PDA, Palm Pilot).

In other preferred embodiments, the digital access device 135 is a digital access point for wireless communication and routing of network packets to other network segments and topologies (e.g,, Wi-Fi). In still further embodiments, the digital access device 135 contains a unique digital signature. In further embodiments, such a digital signature is used for animal identification, animal location, and cross referencing. In still further embodiments, the digital access device 135 operates on low power consumption. In other preferred embodiments, the digital access device 135 is capable of being in a “sleep mode” when not in use. In still further embodiments, the digital access device 135 operates in Class 1 digital radio waves with high sensitivity supporting communications of up to 100 m.

F. Animal Identification Device

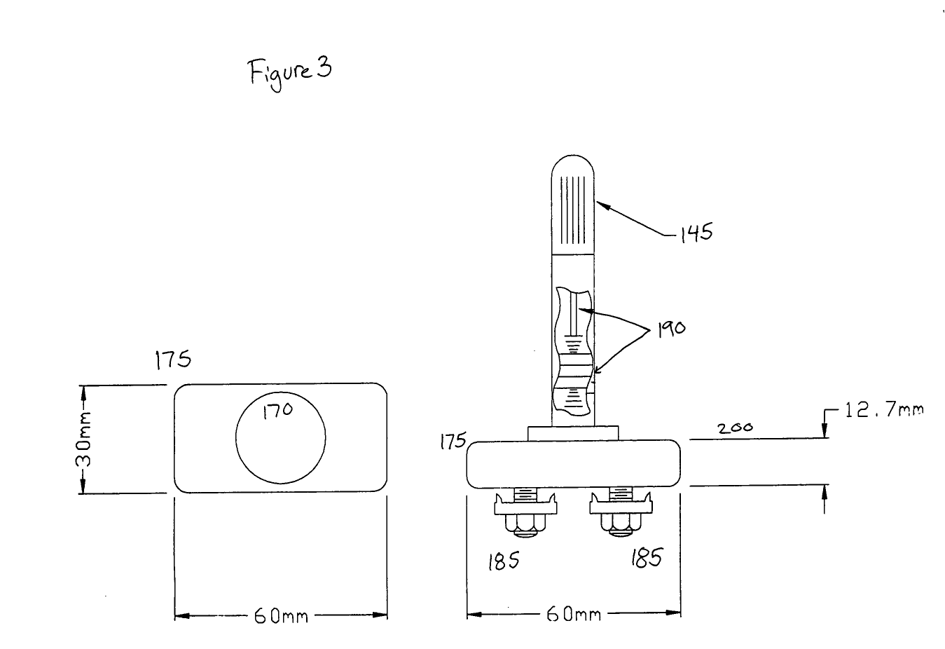

FIG. 3 illustrates an animal identification device 140 embodiment including broadly an elongate shaft 170 (e.g., hollow tube) extending from a central base 175. In some embodiments, the dimensions of the elongate shaft 170 measure less than 30 mm width, and less than 200 mm length. In preferred embodiments, the dimensions of the elongate shaft 170 measure less than 15 mm width, and less than 150 mm length.

In preferred embodiments, the elongate shaft 170 includes an elongate sheath 180 (e.g., protective covering). In further embodiments, the elongate sheath 180 may be made of a polymeric, electrically nonconductive material, like polyethylene or polyurethane or any type of plastic. In other embodiments, the elongate sheath 180 is made with ceramic. In additional embodiments, the elongate sheath 180 is formed with hypo tubing (e.g., stainless steel, titanium).

In further embodiments, the central base 175 interfaces with the elongate shaft 170. In some embodiments the dimensions of the central base 175 measure less than 30 mm height, less than 100 mm width, and less than 50 mm length. In preferred embodiments, the dimensions of the central base 175 measure less than 15 mm height, less than 75 mm width, and less than 40 mm width. In some embodiments, the dorsal side of the central base 175 contains an adherence component 185 (e.g., screws, nuts, snap cap). In preferred embodiments, the adherence component 185 is a screw and a nut. In some embodiments, there is one adherence component 185. In preferred embodiments, there are two adherence components 185. In further preferred embodiments, the adherence component 185 projects less than 20 mm from the central base 175.

In preferred embodiments, the animal identification device 140 is a sealed so as to prevent entry of foreign elements into its interior (e.g., oil, water, dirt, dust). In additional embodiments, the animal identification device 140 is resistant to deterioration due to sunlight or UV rays.

In preferred embodiments of the present invention, the animal identification device 140 is attached to an animal (e.g., cow, goat, or sheep). The location of attachment is anywhere in an animal's body which will allow physical access from outside the animal's body. In preferred embodiments, implantation of the animal identification device 140 is in the animal's ear cartilage. In further preferred embodiments, the animal identification device 140 is implanted in such a manner so as to facilitate easy retrieval. In preferred embodiments, an animal identification device 140 may remain in place beyond 4 years.

In preferred embodiments, the animal identification device 140 comprises a standard BlueTooth radio 190 having an integrated antenna (e.g., a class 2 radio with a range of up to 50 meters). In further preferred embodiments, the Bluetooth radio comprises a BlueCore-02 Bluetooth Chip from CSR. In further preferred embodiments, the BlueTooth Chip is configured to allow 2-axis identification of the location of animals wearing an animal identification device 140. Such tracking systems are available from BlueTags A/Saalborg, Denmark. The Bluetooth radio 190 can receive requests from a digital access device 135 for audible and visual alerts (e.g. battery low). A power source (e.g., battery, not shown) is used to provide energy for the animal identification device 140. In some embodiments, the power source may manually be changed as needed. In further preferred embodiments, each animal identification device 140 is uniquely identified (e.g., an identification number) and serves as a unique identification source for each animal carrying such a device.

In preferred embodiments, the animal identification device 140 also contains an signal device 145 (e.g., light) detectable on the outside of the animal (e.g., outside of animal's ear). In some embodiments, the signal device 145 is an audio device configured to emit different sounds at various volumes for varying amounts of time. In preferred embodiments, the signal device 145 is a light fixture configured to display different colored lights at different intensities for varying amounts of time.

The animal identification device 140 receives messages from the digital access device 135. In preferred embodiments, the animal identification device 140 displays such a message through the signal device 145. In particularly preferred embodiments the signal device 145 will be emit a specific light response (e.g., strobe, colored, flashing) detectable by a dairy farmer upon receipt of a message from the processor 110. The signal device 145 is capable of displaying messages in such a way so as to facilitate animal identification within a herd.

II. Uses of Temperature Detection System

It is contemplated that the present invention be used to maintain the health of a herd of animals through temperature monitoring. In preferred embodiments, the health of a herd of dairy cows are maintained with the present invention.

The present invention may be used in maintaining an animal's reproduction (e.g., estrus) schedule for a large group of animals. The successful breeding of a dairy cow requires precise internal core temperature monitoring, and requires a method of locating the particular cow within a herd. Within an algorithm, the present invention integrates both a particular dairy cow's unique identification number located within an implantable temperature device and that particular dairy cow's core body temperatures over an extended period of time (e.g., weeks). In some embodiments, the processor creates a temperature trend for the particular cow over an extended period of time (e.g., temperature trend from completion of last estrus). In other embodiments, the processor creates an average temperature for the particular cow over a period of time (e.g., temperature average from last estrus). In further embodiments, upon integration, the processor compares the particular dairy cow's temperature information (e.g., temperature trend or average) with standardized cow temperature fluctuation information upon entry into estrus. In other embodiments, upon integration, the processor compares the particular cow's temperature information (e.g., temperature trend or average) with previous temperature fluctuations for that particular cow. If a particular dairy cow's temperature fluctuations indicate the cow is entering, exiting, or not in estrus, the processor sends a signal to the animal identification device indicating the state of the particular cow's estrus. The animal identification device displays this message through the signal device. In preferred embodiments the signal device displays the processor message with a specific light response (e.g., strobe, colored, flashing) detectable by a dairy farmer. Next, the dairy farmer need only locate the dairy cow with a flashing light located in the animal identification device, and proceed with the required reproduction activity (e.g., segregation from herd, mating).

The present invention may be used in maintaining an injection schedule for a large group of animals. In some embodiments, the present invention notifies a dairy farmer of a particular diary cow's injection schedule and assists in locating the particular dairy cow. Dairy cow's receive numerous injections (e.g., hormones, immunizations, BST) which require precise record keeping, and requires a method of locating a particular set of cow's within a herd. Within an algorithm, the present invention integrates both a particular dairy cow's unique identification number located within an implanted transponder and an injection schedule specific for that dairy cow. When a particular cow is scheduled for an injection, the processor communicates this information to the animal identification device. The animal identification device displays this message through the signal device. In preferred embodiments the signal device displays the processor message with a specific light response (e.g., strobe, colored, flashing) detectable by a dairy farmer. Next, the dairy farmer need only locate the dairy cow with a flashing light located in the animal identification device, and proceed with the required injection.

The present invention may be used in maintaining a health maintenance schedule for a large group of animals. Dairy cow's receive numerous health maintenance check-ups (e.g., veterinary checkups, veterinary physical examinations, feet trimming, mating, body scoring) which require precise record keeping, and requires a method of locating a particular set of cow's within a herd. Within an algorithm, the present invention integrates both a particular dairy cow's unique identification number located within an animal identification device and a health maintenance schedule specific for that dairy cow. When a particular cow is scheduled for a form of health maintenance, the processor communicates this information to the animal identification device. The animal identification device displays this message through the signal device. In preferred embodiments the signal device displays the processor message with a specific light response (e.g., strobe, colored, flashing) detectable by a dairy farmer. Next, the dairy farmer need only locate the dairy cow with a flashing light located in the animal identification device, and proceed with the required health maintenance.

The present invention may be used in monitoring the current state of health for a large group of animals. In some embodiments, the present invention notifies a dairy farmer of a particular diary cow's current state of health and assists in locating a particular dairy cow. Temperature fluctuations are often a harbinger of health change (e.g., infection, respiratory disease, infectious contagious intestinal disease, bacterial infection, viral infection, fungal infection) for dairy cows. Within an algorithm, the present invention integrates both a particular dairy cow's unique identification number located within an implantable temperature device and that particular dairy cow's core body temperature over an extended period of time (e.g., weeks). In some embodiments, the processor creates a temperature trend for the particular cow over an extended period of time (e.g., temperature trend from completion of last estrus). In other embodiments, the processor creates an average temperature for the particular cow over a period of time (e.g., temperature average from last estrus). In further embodiments, upon integration, the processor compares the particular dairy cow's temperature information (e.g., temperature trend or average) with a standardized cow's temperature fluctuation information upon entry into particular health changes (e.g., infection, respiratory disease, infectious contagious intestinal disease, bacterial infection, viral infection, fungal infection). In other embodiments, upon integration, the processor compares the particular cow's temperature information (e.g., temperature trend or average) with previous temperature fluctuations for that particular cow. If a particular dairy cow's temperature fluctuations indicate the cow is entering, exiting, or not experience a change in health state, the processor sends a message to the animal identification device. The animal identification device displays this message through the signal device. In preferred embodiments the signal device displays the processor message with a specific light response (e.g., strobe, colored, flashing) detectable by a dairy farmer. Next, the dairy farmer need only locate the dairy cow with a flashing light located in the animal identification device, and proceed with the required state of health activity (e.g., early infection treatment, further veterinary examination).

The present invention is contemplated as a tool for preventive medicine. As such, the present invention may be used to create various databases of information (e.g., temperature trends) based upon a large group of animals. Such databases may be used to monitor the health status of a particular cow, and upon an indicative event (e.g., entrance into estrus, infection), a dairy farmer may initiate appropriate intervention (e.g., breeding, infection treatment, segregation).

The present invention permits the accumulation of temperature trends for an entire herd of dairy cows, and the integration and interpretation of such information. Over an extended period of time (e.g., months) numerous dairy cows within a herd will develop forms of infection. A processor equipped with algorithms and a database may be used to integrate and process temperature trend information for dairy cows who developed forms of infection. The processing of this information permits the development of a database aimed at predicting a cow's propensity for developing a particular form of infection based upon a comparison of a particular dairy cow's temperature trends with the database. In preferred embodiments, databases are created aimed at predicting the development of numerous forms of infection (e.g., infection, respiratory disease, infectious contagious intestinal disease, bacterial infection, viral infection, fungal infection). In further embodiments, infection databases may be used with different herds of cows in a similar preventive medicine capacity.

The present invention also contemplates the development of databases aimed at predicting a dairy cow's estrus state. Over an extended period of time (e.g., months) numerous dairy cows within a herd will enter and exit estrus. A processor equipped with algorithms and a database may integrate and process temperature trend information for dairy cows who enter and exit estrus. The processing of this information permits the development of a database aimed at predicting a cow's propensity for entering estrus. In further embodiments, estrus databases may be used with different herds of cows in a similar capacity.

All publications and patents mentioned in the above specification are herein incorporated by reference. Various modifications and variations of the described devices, compositions, methods, systems, and kits of the invention will be apparent to those skilled in the art without departing from the scope and spirit of the invention. Although the invention has been described in the connection with specific preferred embodiments, it should be understood that the invention as claimed should not be unduly limited to such specific embodiments. Indeed, various modifications of the described modes for carrying out the invention that are obvious to those skilled in the art are intended to be within the scope of the following claims.

We claim:

1. A system comprising: i) an implantable temperature device, ii) a signal receiver, iii) a processor, iv) a digital access device, and v) an animal identification device.

2. The system of claim 1, wherein said implantable temperature device is implantable into an animal.

3. The system of claim 1, wherein said implantable temperature device is implanted into an area of the animal selected from the group consisting of the vulva, eyelid, and ear of said animal.

4. The system of claim 3, wherein said animal is a cow.

5. The system of claim 1, wherein said implantable temperature device, said signal receiver, said processor transmit information through RFID technology.

6. The system of claim 1, wherein said processor, said digital access device, and said animal identification device communicate with a wireless protocol.

7. The system of claim 6, wherein said wireless protocol is Bluetooth.

8. The system of claim 1, wherein said implantable temperature device contains a microchip comprising a unique identification number.

9. The system of claim 1, wherein said animal identification device contains a signal device.

10. The system of claim 1, wherein said signal receiver is positioned in a milking parlor.

11. The system of claim 1, wherein said system is used to monitor the body core temperature of a dairy cow.

12. A method of detecting estrus in an animal comprising the steps of:
a) providing a temperature recording system comprising:
i) at least one animal containing an implantable temperature device comprising a unique identification number; said at least one animal having an animal identification device, wherein said identification device comprises an signal device,
ii) a signal receiver,
iii) a processor, and
b) detecting body core temperature of said at least one animal with said implantable temperature device over an extended period of time;
c) comparing said animal's body core temperature fluctuation over said extended period of time; and
d) identifying a particular animal entering estrus through transmittal of an estrus message from said processor to said animal identification device.

13. The method of claim 12, wherein said at least one animal is at least one cow.

14. The method of claim 12, wherein said transmittal of an estrus message is facilitated by a digital access device.

15. The method of claim 14, wherein said digital access device is a PDA.

16. The method of claim 14, wherein said digital access device utilizes wireless technology.

17. The method of claim 16, wherein said wireless technology is Bluetooth.

18 The method of claim 12, further comprising the step of encoding said processor with said unique identification number for each animal within said at least one animal.

19. The method of claim 12, further comprising the step of encoding said processor with standardized animal temperature fluctuation data upon entry into estrus.
